(19) United States
(12) Patent Application Publication
(10) Pub. No.: US 2011/0281284 A1
(43) Pub. Date: Nov. 17, 2011

(54) NOVEL LIVER CANCER MARKER

(75) Inventors: Hideji Nakamura, Suita-shi (JP); Youichi Yabuuchi, Osaka-shi (JP); Yasukazu Ohmoto, Osaka-shi (JP); Toyoki Mori, Osaka-shi (JP); Masahiro Muraguchi, Osaka-shi (JP); Keiko Oga, Osaka-shi (JP); Fusako Iwata, Osaka-shi (JP); Kaori Murata, Osaka-shi (JP)

(73) Assignees: OTSUKA PHARMACEUTICAL CO., LTD, Chiyoda-ku, Tokyo (JP); HYOGO COLLEGE OF MEDICINE, Nishinomiya-shi, Hyogo (JP)

(21) Appl. No.: 12/673,346

(22) PCT Filed: Aug. 7, 2008
(86) PCT No.: PCT/JP2008/064248

§ 371 Date: Feb. 12, 2010

(30) Foreign Application Priority Data
Aug. 10, 2007 (JP) 2007-209544

(51) Int. Cl. G01N 33/566; C07K 16/22; C07K 14/475

(52) U.S. Cl. 435/7.92; 530/399; 530/389.2; 436/501

(57) ABSTRACT

According to the present invention, HDGF in the blood of patients with chronic liver disease is measured to diagnose whether the patients have liver cancer. A measurement kit for the diagnosis is also provided.

### TECHNICAL FIELD

The present invention relates to a tumor marker for liver cancer, and a method of testing or diagnosing liver cancer. The present invention also relates to a liver cancer diagnostic kit for measuring HDGF-derived polypeptide in the blood.

### BACKGROUND ART

Hepatoma-derived growth factor, HDGF, is a growth factor isolated from a culture supernatant of hepatoma cell line HuH-7. Cloning of the growth factor has been completed, and its DNA sequence has been determined (Non-Patent Document 1). Further, it is known that the growth factor is localized in the nuclear fraction, and that it has an effect of stimulating the proliferation of hepatoma cells, smooth muscle cells, fibroblast cells, and vascular endothelial cells (Non-Patent Documents 2-4). Further, HDGF is also localized in the nuclear fraction of neurons. Contrary to its name, “HDGF” is widely distributed in vivo. Additionally, because of its nutritional factor-like action, the possibility of using HDGF for treatment and diagnosis in patients with nerve diseases has been considered.
[Non-Patent Document 1] Nakamura H. et al., JBC 269: 25143-25149 (1994)
[Non-Patent Document 2] Nakamura H. et al., Clin. Chim. Acta. 183: 273-284 (1989)
[Non-Patent Document 3] Everett A. D. et al., J. Clin. Invest. 105: 567-575 (2000)
[Non-Patent Document 4] Oliver J. A. et al., J. Clin. Invest. 102: 1208-1210 (1998)

### DISCLOSURE OF THE INVENTION

### Object to be Achieved by the Invention

An object of the present invention is to provide a simple method of testing or diagnosing liver cancer, and a liver cancer diagnostic kit necessary for diagnosing liver cancer.

### Means to Achieve the Object

The inventors of the present invention produced an antibody against HDGF, and developed an HDGF measurement method based on the antibody. Further, the inventors determined, by the above-mentioned measurement method, that HDGF exists in the blood, and found that the amount of HDGF was significantly high, particularly in liver cancer patients with chronic liver disease.

The present invention provides a method of diagnosing liver cancer patients, and a diagnostic kit therefor.

Item 1. A liver cancer marker in the blood, the marker being a human HDGF-derived polypeptide that binds with human HDGF-recognizing antibody comprising an amino acid sequence of SEQ ID NO: 1.

Item 2. A method of testing whether a patient has liver cancer, comprising collecting blood from a subject, and detecting the liver cancer marker as defined in Item 1 in the blood sample.

Item 3. The testing method as defined in Item 2, wherein the detection is performed by immunoassay.

Item 4. The testing method as defined in Item 3, wherein the immunoassay is an ELISA.

Item 5. The testing method as defined in Item 3, wherein the subject is a patient with chronic liver disease.

Item 6. A liver cancer diagnostic kit comprising, as a component, at least an antibody that specifically recognizes the marker as defined in Item 1.

### Effects of the Invention

The present invention enables a diagnosis of liver cancer by measuring the HDGF in the blood of patients with chronic liver disease.

Further, the present invention is also able to provide a liver cancer diagnostic kit.

### BRIEF DESCRIPTION OF DRAWINGS

FIG. 1 shows a procedure for ELISA measurement of HDGF.

FIG. 2 shows a standard curve of HDGF. The detection range is 0.31 to 20 ng/ml.

FIG. 3 shows the amount of HDGF in the blood (plasma HDGF) of each patient with liver disease.

Note that HCC indicates a patient with hepatocellular carcinoma, CHC indicates a patient with hepatitis C, CHB indicates a patient with hepatitis B, and LC indicates a patient with liver cirrhosis.

### BEST MODE FOR CARRYING OUT THE INVENTION

Liver diseases include liver cancer, viral hepatitis type B, viral hepatitis type C, cirrhosis, and other various diseases. Damage is inflicted on the liver cells themselves. Therefore, the possibility of liver disease may be determined by measuring the liver cell's contents in the blood. For example, both GOT and GPT are enzymes that are active in the liver cells. When the liver cells are destroyed due to hepatitis and the like, GOT and GPT leak into the blood. Therefore, the GOT and GPT levels in the blood are used as the indices indicative of the degree of liver damage. The GOT and GPT are valuable indices of diseases such as acute hepatitis, chronic hepatitis, etc. in which liver cells are destroyed; however, they are not appropriate as indices of liver cancer, in which the destruction of liver cells is not significant.

HDGF is present in the nuclear fraction. Because it was assumed that HDGF leaks into the blood only after the cells are destroyed, HDGF was expected to be a marker similar to GOT and GPT; however, the inventors of the present invention found, against all expectations, that HDGF, unlike GOT and GPT, is a specific liver cancer marker.

### Liver Cancer Marker

The term “human HDGF” as used in the present specification refers to a growth factor having an amino acid sequence of SEQ ID NO: 1.

The term “human HDGF-recognizing antibody” includes antibodies that recognize a partial peptide produced by decomposition of HDGF insofar as those antibodies recognize human HDGF. The antibodies may be monoclonal or polyclonal. The antibodies may also be antibody fragments insofar as they can bind to antigen. Examples of antibody fragments include scFv, Fab, F(ab)₂, Fv, etc.

The term “human HDGF-derived polypeptide” as used in the present specification includes not only polypeptides that include human HDGF itself, but also a wide range of polypeptides (including epitopes) that can be recognized by antihuman HDGF antibody, such as polypeptides that include human HDGF modified by methylation, amidation, acetylation, etc. in vivo, and polypeptides that are shortened by the enzymatic or chemical decomposition (specifically hydrolysis) of human HDGF.

The liver cancer marker of the present invention includes a human HDGF-derived polypeptide. Liver cancer patients show an increased amount of human HDGF-derived polypeptide in the blood. Samples used for measuring a liver cancer marker include blood, plasma, and serum.

The liver cancer marker of the present invention is expected to be usable as a higher-precision cancer marker by combining the liver cancer marker of the present invention with other conventionally known liver cancer markers such as, for example, AFP (α-fetoprotein), which is a protein produced in the liver and the yolk sac during fetal life and whose increase is observed in about 80% of patients with hepatocellular carcinoma.

### Testing Method

The liver cancer testing method of the present invention is described below in detail.

The present testing method comprises two steps: collecting blood, and detecting a liver cancer marker. First, the sample to be measured is blood. The sample may be either serum or plasma. Additionally, there are no particular limitations on the collection of blood; however, blood is preferably collected during fasting.

There are no particular limitations on the method of detecting or measuring a liver cancer marker insofar as the HDGF-derived polypeptide in the blood can be measured by the method. Preferable examples of such methods include commonly employed biochemical procedures, particularly procedures based on immunological principles. Preferable examples include procedures such as ELISA, Western blotting, and radioimmunoassay (RIA), which employ an antibody against a target substance to be measured. Further, when an antibody is used, the antibody may be either monoclonal or polyclonal. Alternatively, a molecule such as an aptamer, which is derived from a nucleic acid and has affinity for a target substance to be measured, may be used. Further, when contaminants are present in the sample, the target molecule is first purified, or the purity of the sample is increased to an extent where the contaminants have no effect before using the sample for the measurement. Purification methods include gel filtration, ion exchange chromatography, hydrophobic chromatography, affinity chromatography, reversed-phase chromatography, normal phase chromatography, various electrophoresis techniques, ammonium sulfate precipitation, precipitation in an organic solvent, isoelectric precipitation, etc. These methods can be used according to the situation. Needles to say, these are merely examples, and the purification method is not limited to these above-mentioned methods.

The ELISA may be either a direct method or a sandwich method when producing an ELISA kit for measuring HDGF. Measurement can be performed according to conventional methods. Further, in this case, either a monoclonal antibody or polyclonal antibody may be used. For example, in the case of a sandwich method, a primary antibody (anti-HDGF antibody) is usually immobilized on a 96-well plate. A sample derived from a living body (blood, serum, or plasma), which may be diluted, as needed, with an appropriate buffer solution, is added to the plate to allow contact with the antibody for a certain period of time, and thereby the sample is bound to the antibody. After washing the plate with an appropriate buffer solution, a labeled secondary antibody (labeled anti-HDGF antibody) is added for reaction. For example, when the labeling substance is biotin, avidin- (or streptavidin-)labeled peroxidase is added for reaction, and a suitable reactive substrate (for example, TBM) is added for color development. After a certain period of time, measurement is performed at a specific wavelength (450 nm in this case), and the colorimetric determination is thereby carried out. Peroxidase (HRP) labeling, alkaline phosphatase labeling, acid phosphatase labeling, glucose oxidase labeling, tyrosinase labeling, and the like may be used as avidin-labeled antibodies. There are no particular limitations on the substrate, insofar as the substrate is commercially available and commonly used.

Further, a Western blotting method may also be used as an immunological measurement method. In the method of the present invention, electrophoresis is performed on the sample (representative examples of electrophoresis include paper electrophoresis and isoelectric electrophoresis such as SDS-PAGE, PAGE, etc.); the sample is transferred onto a transfer membrane such as a nitrocellulose membrane, PVDF membrane, etc.; a primary antibody against a target molecule to be measured is added to the sample; and a secondary antibody bound to a labeled antibody such as a pigment particle is added to the sample, or, when an labeled enzyme is used, a substrate for the enzyme is added to the sample after the treatment, thereby allowing the target molecule to be visualized. Biotin-labeled antibody is used as a labeled antibody, to which avidin or streptavidin is bound. Or, a peroxidase (HRP)-labeled antibody, an alkaline phosphatase-labeled antibody, an acid phosphatase-labeled antibody, a glucose oxidase-labeled antibody, a tyrosinase-labeled antibody or the like is used as a secondary antibody. There are no particular limitations on the substrate, insofar as the substrate is commercially available and commonly used.

Additionally, the antibody used for measurement can be obtained according to conventional methods. For example, in the case of the production of a polyclonal antibody, warm-blooded animals such as rabbits, sheep, guinea pigs, and chickens are immunized several times with an emulsified substance usually prepared by mixing the above-mentioned target antigen (human HDGF, or its partial peptide or partial polypeptide) with Freund's complete adjuvant. A resulting anti-serum can be obtained according to conventional methods. Further, in the case of chickens, the chickens are immunized with the above-mentioned immunizing antigen several times, IgY is produced in eggs laid by the chickens, and the IgY can be obtained from the yolk of the eggs according to conventional methods.

Further, the antibody can be obtained as a monoclonal antibody. The monoclonal antibody can be obtained, for example, by immunizing mice several times with the above-mentioned immunizing antigen together with Freund's complete adjuvant to induce an antibody, isolating the resulting antibody-producing cells according to conventional methods such as a cell fusion method, and culturing the cells. The thus-obtained antibody can be further purified, as needed, according to conventional methods such as ammonium sulfate precipitation, ion exchange chromatography, affinity chromatography, etc.

### Disease and Subject to be Diagnosed

The present invention can be used for the diagnosis of patients with chronic liver disease, particularly to determine whether their chronic liver disease has progressed to liver cancer. In the case of patients with viral hepatitis or cirrhosis, cancer may be defined as the last stage of the progression of such diseases. For evaluation of the mental condition of the patients or the motivation for treatment, it is important for patients with chronic liver disease to know what pathological stage they are at.

### Diagnostic Kit

Diagnosis of liver cancer in patients with chronic liver disease can be made by measuring the amount of HDGF in the blood. Specifically, the present invention is able to provide an ELISA kit and a kit composition including various reagents for Western blotting. In particular, the kit includes an antibody against HDGF. Besides the antibody, the kit includes albumin such as BSA, secondary antibodies, enzyme substrates (when an enzyme is used as a label), and the like. For example, when the recognizing antibody is a biotin-labeled antibody, the measurement kit may include a peroxidase-labeled avidin as a secondary antibody. Further, the present invention provides a liver cancer diagnostic kit including a substrate for the peroxidase and the like.

### Example

The details of the present invention are described below with reference to an Example. However, the details of the present invention are not limited to the following Example.

### Example 1

### Construction of ELISA Measurement System for HDGF Production of HDGF for Monoclonal Antibody-Antigen and for Standard Preparation

First of all, human HDGF gene is already known, and the gene is represented by SEQ ID NO: 2. Cloning was performed according to conventional methods. Specifically, the region from the initiation codon to the stop codon of HDGF gene was amplified by the PCR method using a human kidney cDNA library. An NdeI site was added to the 5′ end of the forward primer, and an XhoI site was added to the 3′ end of the reverse primer. HDGF cDNA was obtained by cloning a PCR product into pCR-Blunt vector, and confirming the DNA sequence.

Next, the HDGF cDNA of the present invention and a His-tag linker were inserted into pSecTag2 vector, and an expression vector (pSecTag2-HDGF) was thereby constructed. Further, the obtained expression vector (pSecTag2-HDGF) was introduced into CHO-K1 cells, the cells were cultured in the zeocin-containing medium under limiting dilution conditions, and resistant clones were thereby constructed. Further, the presence of HDGF in the culture supernatant was confirmed.

Thereafter, high-producing HDGF clones (CHO/HDGF clone 4) were mass-cultured, culture supernatants were collected, and antigen HDGF was purified.

Specifically, antigen HDGF was purified by Ni resin column chromatography, heparin-Sepharose column chromatography, and resource Q column HPLC to obtain immunogen and a standard preparation.

### Production of Mouse Monoclonal Antibody

Mice were immunized according to conventional methods, and a hybridoma that produces a desired antibody was selected. Specifically, mice were immunized with an emulsion prepared by mixing antigen solution with an equal volume of complete Freund's adjuvant. Immunization was performed every two weeks, for a total of 5 immunizations.

Next, splenocytes were prepared from immunized mice, and mixed with myeloma cells (P3U1) prepared in advance such that the ratio of splenocytes to P3U1 was 5:1 or 2:1. After centrifugal separation, PEG solution was added, while stirring well, to the cell sediment. After the mixture became uniform, it was kept still. The supernatant was removed after centrifugation, and the sediment was suspended in a 10% FCS/RPMI 1640 supplemented with 100 ml of HAT. The cells were seeded onto a 24-well culture plate (coaster) at 1 ml/well, and cultured at 37° C. in 5% CO₂ to allow cell fusion.

Positive hybridomas were cloned in the following manner.

A culture supernatant was removed from each well one week to 10 days after cell fusion, and antibody screening was performed. The culture supernatant was reacted on a 96-well plate (NUNC) to which HDGF antigen was immobilized to check for the presence of antibodies by ELISA.

For the production of ascites, mice were intraperitoneally injected with pristane (2,6,10,14-etramethylpentadecan) at 0.5 ml/body at least 3 days in advance of cell injection. Hybridoma cells were suspended in 1.5 ml of PBS, and 3 mice were each injected intraperitoneally with 0.5 ml of hybridoma cells. Ascites was collected from the mice when the abdomens of the mice injected with the cells were largest.

Monoclonal antibody was purified from ascites by applying the ascites to protein A column, and the concentration thereof was verified by checking the purity by SDS-PAGE.

### Production of Rabbit Polyclonal Antibody

HDGF antigen solution was mixed with complete Freund's adjuvant to prepare an emulsion, and rabbits were intradermally immunized on the back at several sites (1 mL/site).

Immunization was performed every two weeks. Blood was partially collected from the earlobe one week after three immunizations. The antibody titer of the serum was checked by ELISA, and four more immunizations were repeated. All of the blood was collected from the carotid artery one week after the final immunization (blood collection with heparin). The blood was subjected to centrifugal separation to obtain plasma.

Note that for the production of a polyclonal antibody that uses C-terminal peptide as an antigen, a peptide having a Cys at the N-terminal of the C-terminal peptide (amino acids at positions 231 to 240 from the N-terminal) was synthesized, purified, and coupled to KLH as the antigen (KLH-C-APGIRDHESL).

### Establishment of ELISA

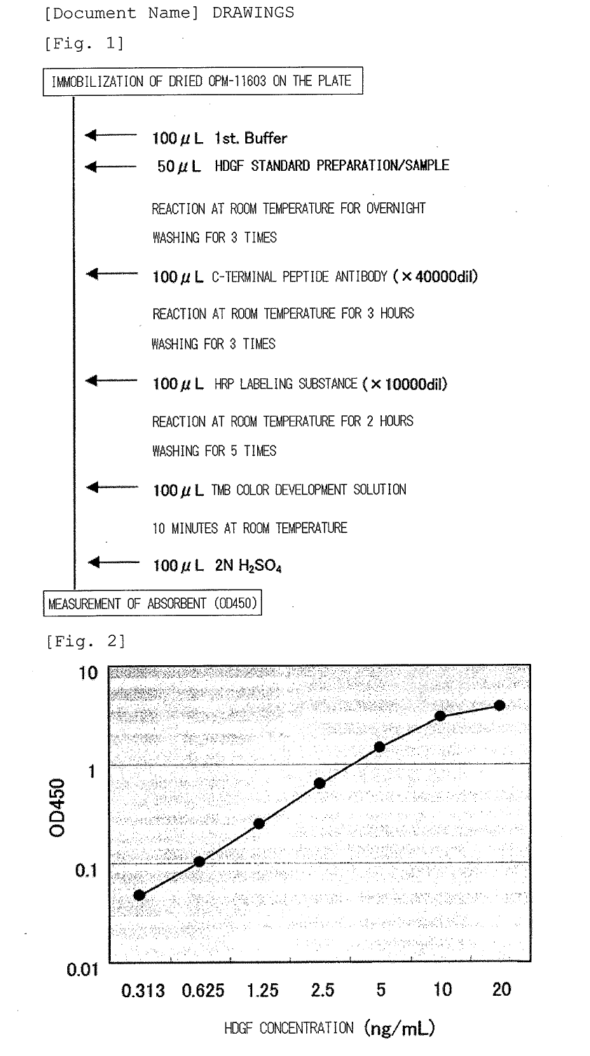

For ELISA for HDGF, monoclonal antibody OPM-11617 was diluted to 5 μg/mL in 0.1 M of NaCO₃, immobilized in each well, and blocked with 0.1% BSA/PBS. 0.1% BSA/PBS/0.05% Tween-20 was used as a first buffer, which was placed in a 96-well plate in advance. The standard HDGF was diluted using 20 mg/mL of BSA/PBS. 7 two-fold serial dilutions from 20 ng/ml were prepared as standard preparations having different concentrations, and a blank was used to determine the calibration curve. A volume of 50 μL of standard preparation and sample was added to the well and allowed to react at room temperature for 24 hours. After washing the well, C-terminal antibody (100 μL) diluted 40,000-fold by 0.1% BSA/PBS/0.05% Tween-20 was added to the well, and allowed to react at room temperature for 2 hours. Further, the plate was washed, and HRP-labeled avidin diluted 10,000 times was added to each well and allowed to react at room temperature for 2 hours. After washing the plate, TMB solution was added to the plate and allowed to react at room temperature for 10 minutes. The reaction was stopped with 2N sulfuric acid. The absorbance was set to 450 and measured at the wavelength. FIG. 1 shows a procedure for measurement.

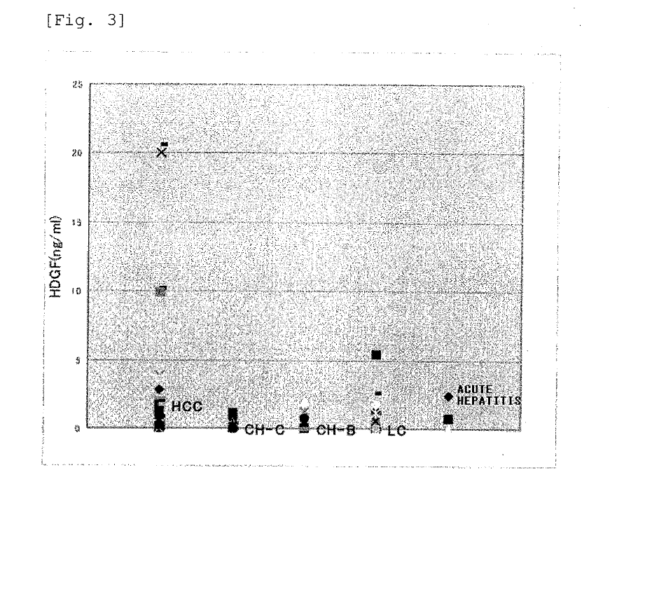

FIG. 2 shows a calibration curve of HDGF. The measurement range is from 0.31 ng/ml to 20 ng/mL. The measurement of HDGF in cell culture was made possible.

Measurement of HDGF in Serum of Patients with Chronic Liver Disease

Blood was collected from patients with liver cancer (61 people), viral hepatitis type C (61 people), viral hepatitis type B (9 people), cirrhosis (55 people), and acute hepatitis (3 people) during fasting, and the value of HDGF in plasma was measured using the above-described established ELISA system. The results show that the amount of HDGF is significantly high in plasma from patients with liver cancer (FIG. 3, Table 1).

TABLE 1

| | Liver | Hepatitis | Hepatitis | Liver | Acute |
|---|---|---|---|---|---|
| | Cancer | C | B | Cirrhosis | Hepatitis |
| Negative | 25 | 54 | 4 | 40 | 1 |
| Positive | 36 | 7 | 5 | 15 | 2 |
| Total/240 | 61 | 61 | 9 | 55 | 3 |

1. A liver cancer marker in the blood, the marker being a human HDGF-derived polypeptide that binds with human HDGF-recognizing antibody, wherein the human HDGF comprises an amino acid sequence of SEQ ID NO: 1.

2. A method of testing whether a patient has liver cancer, comprising collecting blood from a subject, and detecting the liver cancer marker as defined in claim 1 in the blood sample.

3. The testing method as defined in claim 2, wherein the detection is performed by immunoassay.

4. The testing method as defined in claim 3, wherein the immunoassay is an ELISA.

5. The testing method as defined in claim 3, wherein the subject is a patient with chronic liver disease.

6. A liver cancer diagnostic kit comprising, as a component, at least an antibody that specifically recognizes the marker as defined in claim 1.


SEQUENCE LISTING (2 sequences)

SEQ ID NO: 1 - protein, 240 aa, Homo sapiens
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEAAEGDGDKKGNAEGSSDEEGKLVIDEPAKEKNEKGALKRRAGDLLEDSPKRPKEAENPEGEEKEAATLEVERPLPMEVEKNSTPSEPGSGRGPPQEEEEEEDEEEEATKEDAEAPGIRDHESL

SEQ ID NO: 2 - DNA, 2376 bp, Homo sapiens
gaggaggagtggggaccgggcggggggtggaggaagaggcctcgcgcagaggagggagcaattgaatttcaaacacaaacaactcgacgagcgcgcacccaccgcgccggagccttgccccgatccgcgcccgccccgtccgtgcggcgcgcgggcggagacgccgtggccgcgccggagctcgggccgggggccaccatcgaggcgggggccgcgcgagggccggagcggagcggcgccgccaccgccgcacgcgcaaacttgggctcgcgcttcccggcccggcgcggagcccggggcgcccggagccccgccatgtcgcgatccaaccggcagaaggagtacaaatgcggggacctggtgttcgccaagatgaagggctacccacactggccggcccggattgacgagatgcctgaggctgccgtgaaatcaacagccaacaaataccaagtcttttttttcgggacccacgagacggcattcctgggccccaaagacctcttcccttacgaggaatccaaggagaagtttggcaagcccaacaagaggaaagggttcagcgaggggctgtgggagatcgagaacaaccctactgtcaaggcttccggctatcagtcctcccagaaaaagagctgtgtggaagagcctgaaccagagcccgaagctgcagagggtgacggtgataagaaggggaatgcagagggcagcagcgacgaggaagggaagctggtcattgatgagccagccaaggagaagaacgagaaaggagcgttgaagaggagagcaggggacttgctggaggactctcctaaacgtcccaaggaggcagaaaaccctgaaggagaggagaaggaggcagccaccttggaggttgagaggccccttcctatggaggtggaaaagaatagcaccccctctgagcccggctctggccgggggcctccccaagaggaagaagaagaggaggatgaagaggaagaggctaccaaggaagatgctgaggccccaggcatcagagatcatgagagcctgtagccaccaatgtttcaagaggagcccccaccctgttcctgctgctgtctgggtgctactggggaaactggccatggcctgcaaactgggaacccctttcccaccccaacctgctctcctcttctactcacttttcccactccaagcccagcccatggagattgacctggatggggcaggccacctggctctcacctctaggtccccatactcctatgatctgagtcagagccatgtcttctccctggaatgagttgaggccactgtgttccttccgcttggagctattttccaggcttctgctggggcctgggacaactgctcccacctcctgacacccttctcccactctcctaggcattctggacctctgggttgggatcaggggtaggaatggaaggatggagcatcaacagcagggtgggcttgtggggcctgggaggggcaatcctcaaatgcggggtgggggcagcacaggagggcggcctccttctgagctcctgtcccctgctacacctattatcccagctgcctagattcagggaaagtgggacagcttgtaggggaggggctcctttccataaatccttgatgattgacaacacccatttttccttttgccgaccccaagagttttgggagttgtagttaatcatcaagagaatttggggcttccaagttgttcgggccaaggacctgagacctgaagggttgactttacccatttgggtgggagtgttgagcatctgtccccctttagatctctgaagccacaaataggatgcttgggaagactcctagctgtcctttttcctctccacacagtgctcaaggccagcttatagtcatatatatcacccagacataaaggaaaagacacattttttaggaaatgtttttaataaaagaaaattacaaaaaaaaattttaaagacccctaaccctttgtgtgctctccattctgctccttccccatcgttgcccccatttctgaggtgcactgggaggctccccttctatttggggcttgatgactttctttttgtagctggggctttgatgttccttccagtgtcatttctcatccacataccctgacctggccccctcagtgttgtcaccagatctgatttgtaacccactgagaggacagagagaaataagtgccctctcccaccctcttcctactggtctctctatgcctctctacagtctcgtctcttttaccctggcccctctcccttgggctctgatgaaaaattgctgactgtagctttggaagtttagctctgagaaccgtagatgatttcagttctaggaaaataaaacccgttgattact